(19) United States
(12) Patent Application Publication
(10) Pub. No.: US 2019/0060330 A1
(43) Pub. Date: Feb. 28, 2019

(54) APPLICATION OF 4-HYDROXY SALICYLANILIDE IN PREPARATION OF ANTI-MYELOMA OR ANTI-LYMPHOMA DRUGS

(71) Applicant: Jumei SHI, Shanghai (CN)

(72) Inventors: Jumei SHI, Shanghai (CN); Weiliang ZHU, Shanghai (CN); Jimin SHAO, Shanghai (CN); Guang YANG, Shanghai (CN); Zhijian XU, Shanghai (CN); Xia LIU, Shanghai (CN); Lu GAO, Shanghai (CN); Bo LI, Shanghai (CN); Xinhuan CHEN, Shanghai (CN)

(21) Appl. No.: 16/093,107

(22) PCT Filed: May 26, 2016
(86) PCT No.: PCT/CN2016/083463

§ 371 Date: Oct. 11, 2018

(30) Foreign Application Priority Data
Apr. 11, 2016 (CN) 201610220405.5

(51) Int. Cl. A61K 31/609; A61P 35/00

(52) U.S. Cl. CPC A61K 31/609; A61K 9/0019; A61P 35/00

(57) ABSTRACT

The present invention provides an application of 4-hydroxyl salicylanilide in preparation of drugs for preventing or treating lymphoma or multiple myeloma.

### TECHNICAL FIELD

The present invention relates to the technical field of anti-cancer drugs and, specifically, relates to the application of 4-hydroxy salicylanilide in preparation of anti-myeloma or anti-lymphoma drugs.

### BACKGROUND OF INVENTION

Multiple myeloma (MM) is a malignant disease with abnormal proliferation of clonal plasmocytes. It is the second most common malignant tumor of blood system, accounting for about 10% of hematological malignancies. It mostly occurs in the middle-aged and elderly population and is still incurable. The median survival time is 4 to 5 years. The main methods of traditional treatment for multiple myeloma are chemotherapy and hematopoietic stem cell transplantation, while it is hard to maintain the clinical efficacy. In the past 10 years, with the emergence of new drugs such as proteasome inhibitors including bortezomib, immunomodulators including thalidomide and lenalidomide, the complete remission rate and overall survival rate of the patients suffered from multiple myeloma have increased significantly. Meanwhile, however, the following deficiencies still exist: first, the effective rate of single drug among these drugs in relapsed/refractory patients is only 25% to 50%; second, although the disease-free survival time is prolonged, most patients will eventually relapse, and significant drug resistance occurs; third, the use of drugs is limited by some serious side effects, such as neuritis. Therefore, the development and verification of new therapeutic drugs remains an important challenge for the treatment of multiple myeloma at present.

Lymphoma is one of the most commonly seen malignant tumors of blood system, and it ranks the 8th among the common malignant tumors in China, and the incidence is still increasing in recent years. New treatment regimens such as chemotherapy, monoclonal antibodies, and cellular immunotherapy have significantly improved the survival of patients suffered from lymphoma. In particular, the breakthrough was made for the treatment of lymphoma, especially for CD20-positive B-cell lymphoma, by the emergence of rituximab, which is more efficient with longer remission duration and significantly improved prognosis. However, the relapse or resistant rate of lymphoma is still high. Therefore, it remains necessary to further develop new drugs to improve the therapeutic effect and cure rate of lymphoma.

The formula of 4-hydroxy salicylanilide is shown as follows:

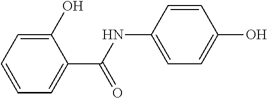 [SMILES: O=C(Nc1ccc(O)cc1)c1ccccc1O]

The compound has a molecular formula of C₁₃H₁₁NO₃, is white powder, and has a molecular weight of 229.24 and CAS number thereof is 526-18-1. The compound, commonly known as sulphate, is currently used in hepatobiliary diseases. The mechanism thereof is similar to that of the dehydrocholic acid, which can increase hepatic blood flow, improve liver function, and significantly increase the water content in bile. The choleretic effect is stronger than dehydrocholic acid, which can relax the Oddi sphincter. It has been shown in studies that 4-hydroxy salicylanilide is an inhibitor of Ribonucleotide reductase (RR).

### SUMMARY OF INVENTION

The present invention provides a use of 4-hydroxy salicylanilide in preparing medicament for preventing or treating lymphoma.

As a preferred embodiment of the present invention, the lymphoma is non-Hodgkin's lymphoma.

As a preferred embodiment of the present invention, the non-Hodgkin's lymphoma is a B cell lymphoma.

As a preferred embodiment of the present invention, the B cell lymphoma is a diffuse large B cell lymphoma.

As a preferred embodiment of the present invention, the medicament comprises 4-hydroxy salicylanilide and a pharmaceutically acceptable carrier.

As a preferred embodiment of the present invention, the medicament is prepared into a tablet, granule, injection or capsule.

The invention also provides a use of 4-hydroxy salicylanilide for the preparation of a medicament for preventing or treating multiple myeloma.

As a preferred embodiment of the present invention, the medicament comprises 4-hydroxy salicylanilide and a pharmaceutically acceptable carrier.

As a preferred embodiment of the present invention, the medicament is prepared into a tablet, granule, injection or capsule.

It has been found from the experiments and the studies that this compound can effectively inhibit the growth of multiple myeloma and lymphoma cells in vitro. In vivo, the compound can effectively inhibit the growth of multiple myeloma and lymphoma in mice, and can be developed into a medicament for preventing and treating multiple myeloma and lymphoma. Pharmaceutically acceptable carriers can be mixed with the compound to prepare conventional dosage forms such as a tablet, a granule, an injection or the like.

The advantages of Invention include:

1. The present invention explores a new medical use for the known drug of 4-hydroxy salicylanilide and opens up a new field of application.

2. 4-hydroxy salicylanilide has high cytotoxic activity against multiple myeloma and lymphoma cells.

3. 4-hydroxy salicylanilide is a traditional liver-protecting drug with high safety, indicating that it has a good prospect for medical use in the field of cancer therapy.

### DESCRIPTION OF FIGURES

FIG. 1 to FIG. 5 show the inhibition curves of 4-hydroxy salicylanilide on multiple myeloma cells (H929 cells, OPM2 cells, U266 cells, OCI-MY5 cells, RPMI 8266 cells).

FIG. 6 and FIG. 7 show animal experiments in which 4-hydroxy salicylanilide inhibits multiple myeloma.

FIG. 8 to FIG. 14 show the inhibition curves of 4-hydroxy salicylanilide on lymphoma cells (SUDHL-4 cells, OCI-LY1 cells, OCI-LY8 cells, DB cells, NU-DUL-1 cells, U2932 cells, TMD8 cells).

FIG. 15 to FIG. 16 show animal experiments in which 4-hydroxy salicylanilide inhibits lymphoma.

### DETAILED EMBODIMENT

The detailed embodiments provided by the present invention will be described in detail below with reference to the attached figures.

### Example 1: Killing Activity Against Human Multiple Myeloma Cells

### 1. Experiment Materials

(1) Cell lines: human multiple myeloma cells (H929 cells, OPM2 cells, U266 cells, OCI-MY5 cells, RPMI 8266 cells) (from ATCC of USA. The cells were passaged and preserved in the applicant's laboratory), were cultured in a 1640 culture medium (containing 10% fetus bovine serum).

(2) Main reagents: 1640 culture medium (Gibco Co., USA), fetus bovine serum (Gibco Co., USA), 4-hydroxy salicylanilide (Shanghai Titan Chemical Co., Ltd, CN), Cell Counting Kit-8 (CCK8, Dojindo Laboratories Co., Ltd, JP).

(3) Main instruments: carbon dioxide incubator (Thermo Forma Co., USA), Automatic microplate reader (Bio-TEK, Elx800).

### 2. Experiment Methods

(1) Cell Culture

The cells were cultured in 1640 medium (containing 10% fetal bovine serum, pH 7.2) supplemented with 2 mmol/L glutamine. The cells were cultured in a cell culture incubator at 37° C., 5% CO₂.

(2) Determination of Cytotoxicity of Each Drug by CCK8 Kit

A single-cell suspension of human multiple myeloma cells (H929 cells, OPM2 cells, U266 cells, OCI-MY5 cells, RPMI 8266 cells) was taken for cell counting, and the cell concentration was adjusted to 2×10⁵ cells/mL. 95 μL of the above cell suspension was added into each well of a 96-well culture plate, and then 5 μL of the drug prepared with the medium was added at different concentrations, while the same volume of culture medium was added in the control group. Three parallel wells were set in each group. The plate was subjected to continuous culture for 72 h. 2 hours before the end of the culture, 10 μL of CCK8 reagent was added into each well, and the plate was continued to culture in a CO₂ incubator. After 2 hours, the OD value of each well at 450 nm was detected by an automatic microplate reader. Cell viability and inhibition rate were calculated: cell viability (%)=(OD mean of experimental well/OD mean of control well)×100%. Cell inhibition rate (%)=100%−cell viability (%). The fitting function was used to determine the IC50 of the drug concentration when the cell growth was inhibited by 50%. The experiment was in triplicate.

### 3. Experiment Results

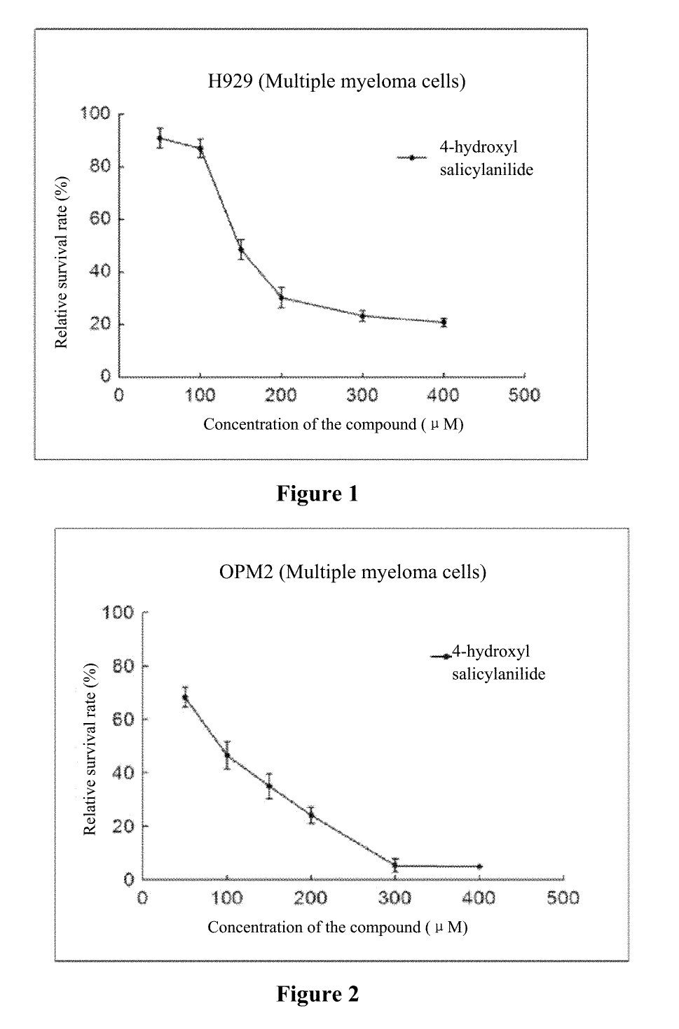

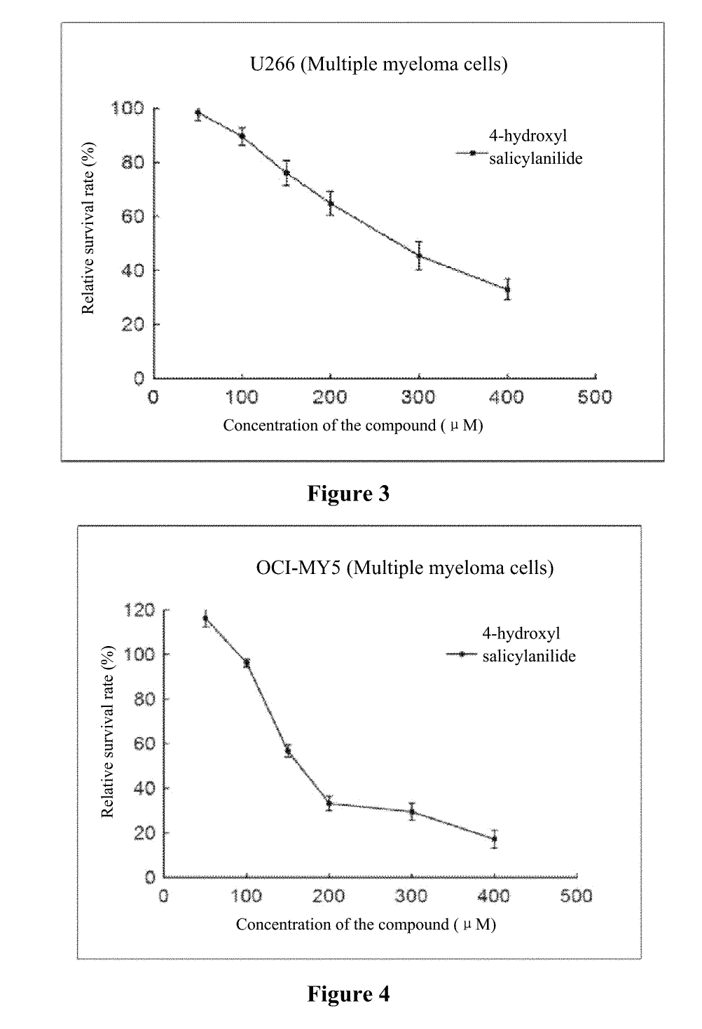

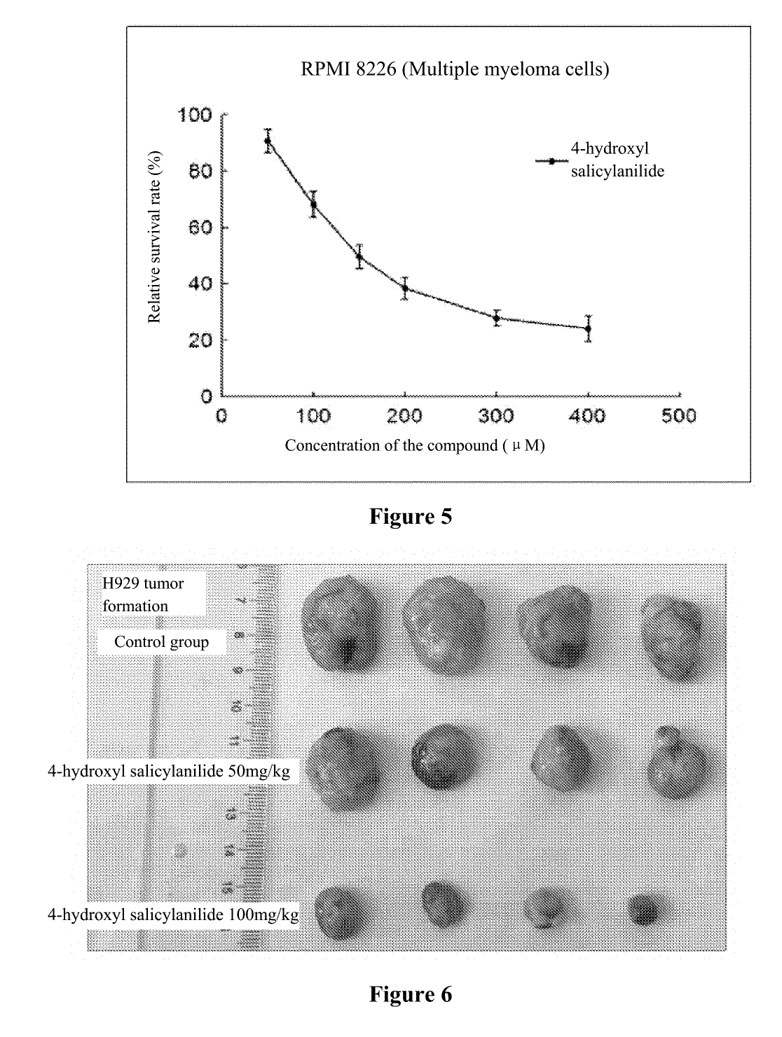

The experiment results are shown in FIG. 1 to FIG. 5.

Conclusion: 4-hydroxy salicylanilide has cytotoxic activity against human multiple myeloma cells. The IC50 values for H929 cells, OPM2 cells, U266 cells, OCI-MY5 cells and RPMI 8266 cells are 179 μM, 84 μM, 271 μM, 211 μM and 170 μM, respectively.

### Example 2: Animal Experiment for Multiple Myeloma

### 1. Experiment Materials

(1) Cell line: human multiple myeloma cells (H929 cells) (from ATCC, USA. The cells were passaged and preserved in the applicant's laboratory), were cultured in a 1640 culture medium (containing 10% fetus bovine serum).

(2) Experimental animals: Male BALB/C nude mice (4-6 week old, purchased from Shanghai Sippr-BK laboratory animal Co. Ltd), were housed in an SPF condition (Center Laborotory Animal Room, Shanghai Tenth People's Hospital).

### 2. Experiment Methods

(1) Cell Culture

The cells were cultured in 1640 a 1640 culture medium (containing 10% fetus bovine serum, pH 7.2) supplemented with 2 mmol/L glutamine. The resultant was cultured in a incubator at 37° C. under 5% CO₂ environment.

(2) Animal Experiments

The 1640 medium containing 3.5×10⁶ H929 cells was injected subcutaneously into the right axilla of the nude mice, and when the tumors grew to a measurable size, the mice were randomly assigned into the control group and the administration group. The nude mice in the administration group were injected through caudal vein with 4-hydroxy salicylanilide at 50 mg/kg and 100 mg/kg per day. The nude mice in the control group were injected with the same volume of solvent (200 μL, 5% DMSO+4% castor oil+91% normal saline). The tumor sizes were measured every two days (measuring the length and width of the tumor, tumor volume=0.5×(width)²×length). The mice were sacrificed after a 22-day administration and the tumors were taken for taking photos. The results are shown in Table 1.

TABLE 1
The in vivo results of animal experiments using 4-hydroxy salicylanilide

| | | Administration time (day) | | | | | | | | | | | |
|---|---|---|---|---|---|---|---|---|---|---|---|---|---|
| | | 0 | 2 | 4 | 6 | 8 | 10 | 12 | 14 | 16 | 18 | 20 | 22 |
| | No. | Volume (cm³) | | | | | | | | | | | |
| Control | 1 | 0.08 | 0.17 | 0.29 | 0.49 | 0.72 | 1.05 | 1.42 | 1.91 | 2.51 | 3.07 | 3.73 | 4.80 |
| group | 2 | 0.07 | 0.12 | 0.22 | 0.45 | 0.78 | 1.25 | 1.83 | 2.22 | 2.68 | 3.29 | 4.15 | 4.94 |
| | 3 | 0.09 | 0.18 | 0.34 | 0.54 | 0.74 | 1.11 | 1.60 | 1.92 | 2.43 | 3.03 | 3.43 | 3.80 |
| | 4 | 0.08 | 0.15 | 0.28 | 0.45 | 0.68 | 0.92 | 1.20 | 1.65 | 2.02 | 2.58 | 3.20 | 4.06 |
| | Mean | 0.08 | 0.16 | 0.28 | 0.48 | 0.73 | 1.08 | 1.51 | 1.93 | 2.41 | 2.99 | 3.63 | 4.40 |
| | SD | 0.01 | 0.02 | 0.04 | 0.03 | 0.04 | 0.12 | 0.23 | 0.20 | 0.24 | 0.26 | 0.36 | 0.48 |
| 4-hydroxy | 1 | 0.08 | 0.15 | 0.24 | 0.38 | 0.58 | 0.77 | 0.99 | 1.20 | 1.53 | 2.08 | 2.59 | 3.39 |
| salicylanilide | 2 | 0.11 | 0.17 | 0.25 | 0.38 | 0.50 | 0.63 | 0.78 | 1.15 | 1.47 | 1.81 | 2.22 | 2.44 |
| (50 mg/kg) | 3 | 0.10 | 0.15 | 0.22 | 0.30 | 0.37 | 0.64 | 0.80 | 0.97 | 1.31 | 1.54 | 1.77 | 2.19 |
| | 4 | 0.08 | 0.11 | 0.14 | 0.27 | 0.37 | 0.49 | 0.74 | 1.03 | 1.35 | 1.71 | 2.12 | 2.89 |
| | Mean | 0.10 | 0.15 | 0.21 | 0.33 | 0.45 | 0.63 | 0.83 | 1.09 | 1.41 | 1.79 | 2.17 | 2.72 |
| | SD | 0.01 | 0.02 | 0.04 | 0.05 | 0.09 | 0.10 | 0.09 | 0.09 | 0.09 | 0.20 | 0.29 | 0.46 |
| 4-hydroxy | 1 | 0.10 | 0.12 | 0.13 | 0.17 | 0.26 | 0.39 | 0.53 | 0.77 | 0.93 | 1.20 | 1.27 | 1.39 |
| salicylanilide | 2 | 0.08 | 0.09 | 0.14 | 0.15 | 0.20 | 0.23 | 0.31 | 0.42 | 0.49 | 0.57 | 0.63 | 0.71 |
| (100 mg/kg) | 3 | 0.04 | 0.08 | 0.13 | 0.17 | 0.25 | 0.31 | 0.39 | 0.44 | 0.50 | 0.57 | 0.65 | 0.80 |
| | 4 | 0.07 | 0.08 | 0.10 | 0.13 | 0.17 | 0.23 | 0.25 | 0.27 | 0.32 | 0.31 | 0.34 | 0.40 |
| | Mean | 0.07 | 0.09 | 0.13 | 0.16 | 0.22 | 0.29 | 0.37 | 0.47 | 0.56 | 0.66 | 0.72 | 0.82 |
| | SD | 0.02 | 0.01 | 0.01 | 0.02 | 0.04 | 0.07 | 0.11 | 0.18 | 0.22 | 0.32 | 0.34 | 0.36 |

Note: The results in the table indicated that 4-hydroxy salicylanilide could significantly inhibit the tumor growth in animals. At day 0, there was no difference in tumor tissue volume between the two groups. P>0.05 (pairwise comparison). At day 22, the tumor tissue volumes of each group were significantly different. When compared the control group with, the 4-hydroxy salicylanilide 50 mg/kg group had a P<0.01; when compared with 4-hydroxy salicylanilide 50 mg/kg group, the 4-hydroxy salicylanilide 100 mg/kg group had a P<0.01. Moreover, 4-hydroxy salicylanilide inhibited tumor growth in animals in a dose-dependent manner. The 4-hydroxy salicylanilide 100 mg/kg group inhibited tumor growth more significantly than the 50 mg/kg group.

### 3. Experiment Results

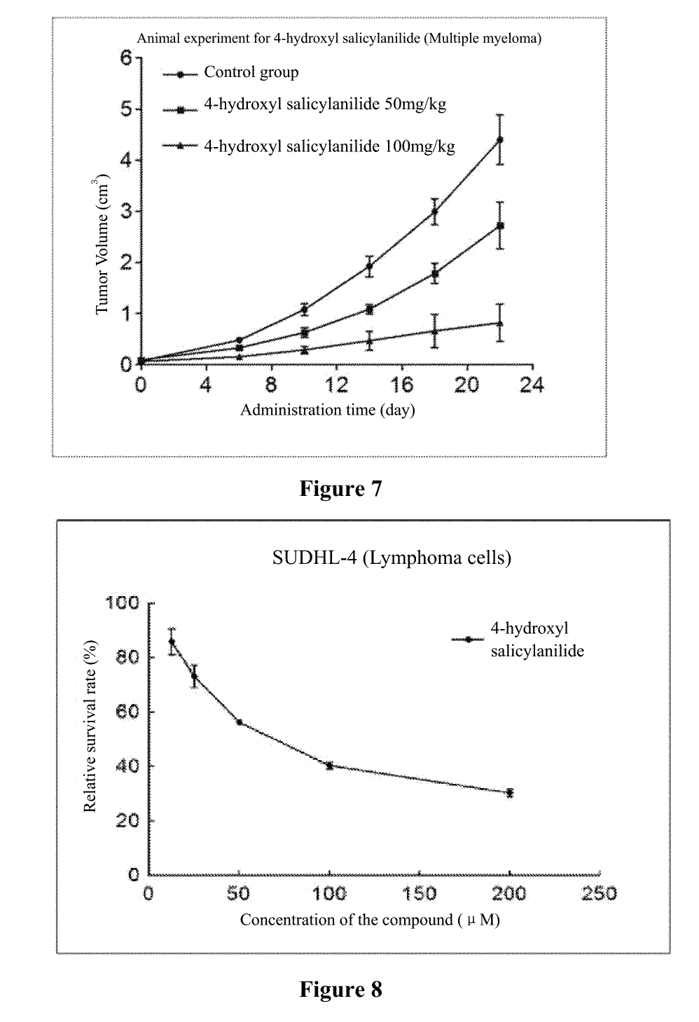

The experiment results are shown in FIG. 6 and FIG. 7.

Conclusion: 4-hydroxy salicylanilide is effective in inhibiting the growth of multiple myeloma in nude mice.

Table 1. The in vivo results of animal experiments using 4-hydroxy salicylanilide.

### Example 3: Killing Activity Against Human Lymphoma Cells

### 1. Experiment Materials

Cell lines: human lymphoma cells (SUDHL-4 cells, OCI-LY1 cells, OCI-LY8 cells, DB cells, NU-DUL-1 cells, U2932 cells, TMD8 cells)(from ATCC. USA. The cells were passaged and preserved in the applicant's laboratory). IMDM (Gibco Co., USA). DMEM (low sugar) (Gibco Co., USA). The rest were the same as in Example 1.

### 2. Experiment Methods

OCI-LY8 cells were cultured in IMDM. U2932 cells were cultured in DMEM (low sugar). The rest were the same as in Example 1.

### 3. Experiment Results

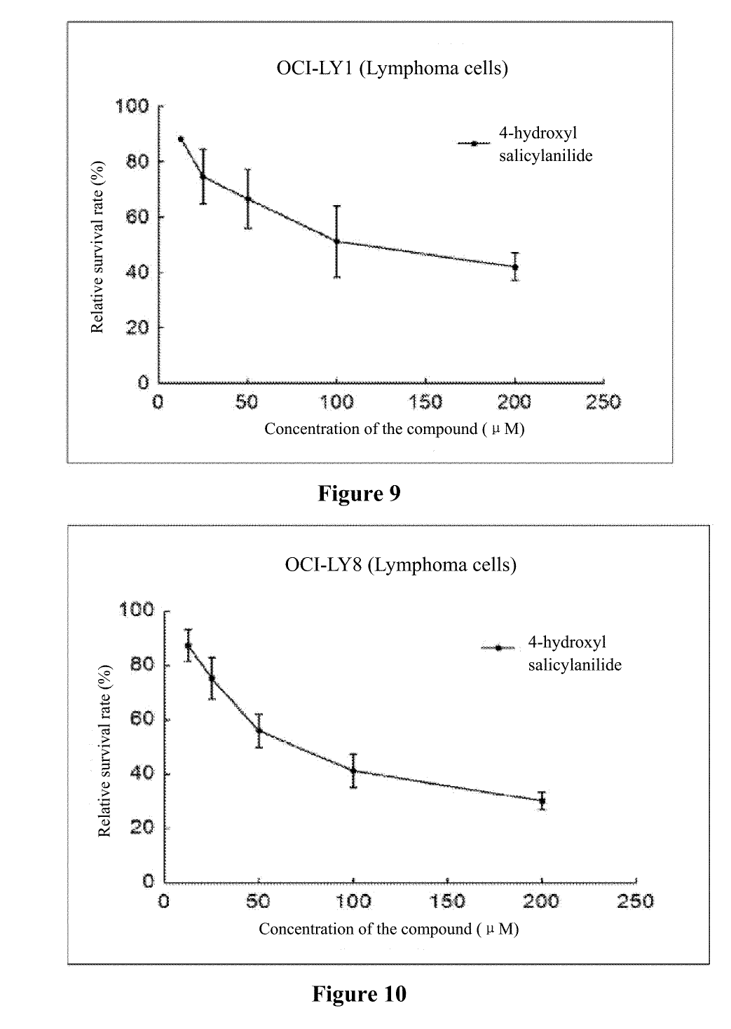

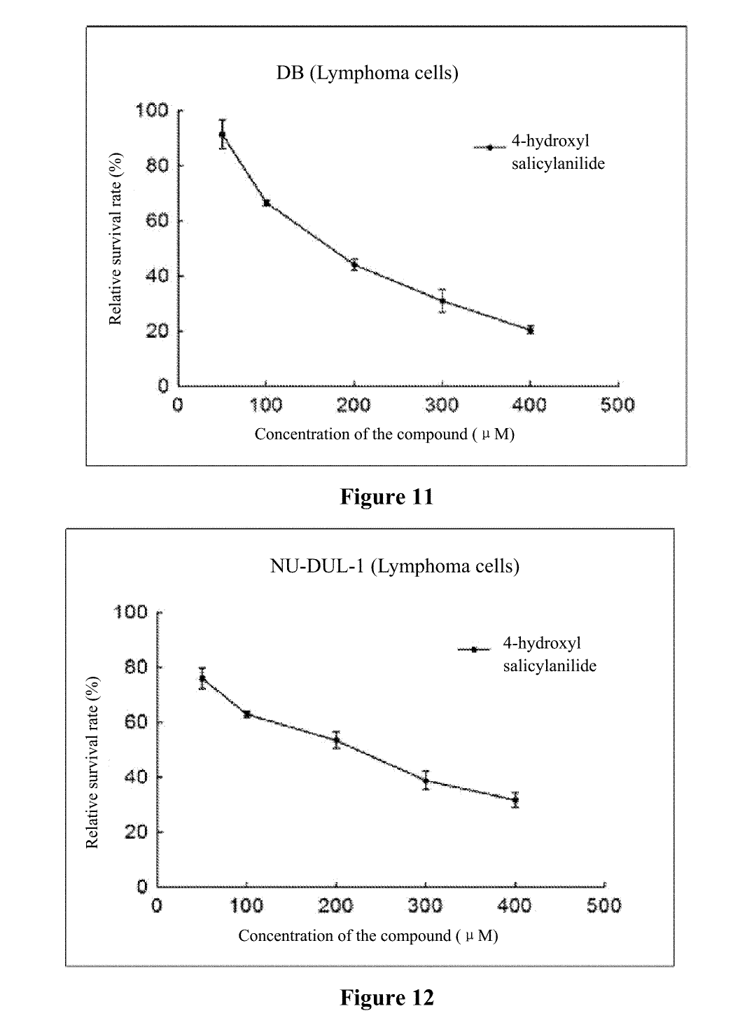

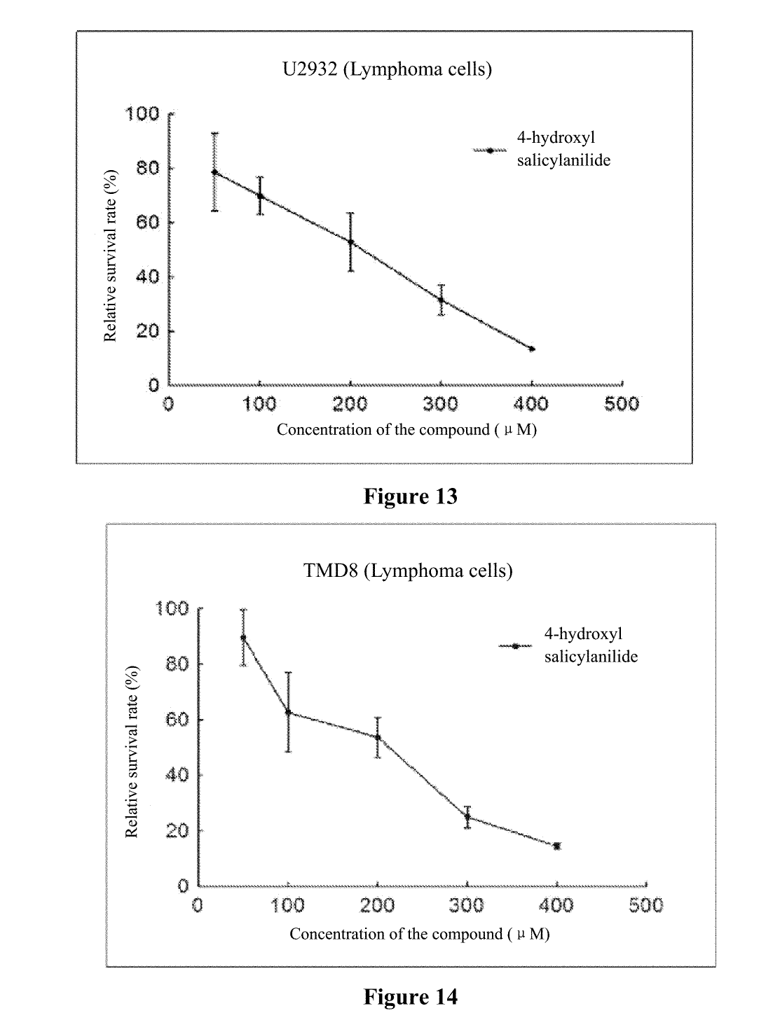

Experiment data is shown in FIG. 8 to FIG. 14.

Conclusion: 4-hydroxy salicylanilide has cytotoxic activity against human lymphoma cells. The IC50 for SUDHL-4 cells, OCI-LY1 cells, OCI-LY8 cells, DB cells, NU-DUL-1 cells, U2932 cells and TMD8 cells are 73 μM, 117 μM, 76 μM, 178 μM, 188 μM, 156 μM, and 164 μM, respectively.

### Example 4: Animal Experiment Against Lymphoma

### 1. Experiment Materials

(1) Cell lines: human lymphoma cells (OCI-LY8 cells) (from ATCC, USA. The cells were passaged and preserved in the applicant's laboratory) were cultured in IMDM (containing 10% fetus bovine serum).

(2) Experimental animals: referring to Example 2.

### 2. Experiment Methods

(1) Cell culture: referring to Example 3

(2) Animal experiments

The IMDM containing 3×10⁶ OCI-LY8 cells was injected subcutaneously into the right axilla of the nude mice, and when the tumors grew to a measurable size, the mice were randomly assigned into the control group and the administration group. The nude mice in the administration group were injected through caudal vein with 4-hydroxy salicylanilide at 60 mg/kg ever other day. The nude mice in the control group were injected with the same volume of solvent (200 μL, 5% DMSO+4% castor oil+91% normal saline). The tumor sizes were measured every two days (measuring the length and width of the tumor, tumor volume=4π/3×(width/2)²×(length/2)). The mice were sacrificed after a 18-day administration and the tumors were taken for taking photos.

### 3. Experiment Results

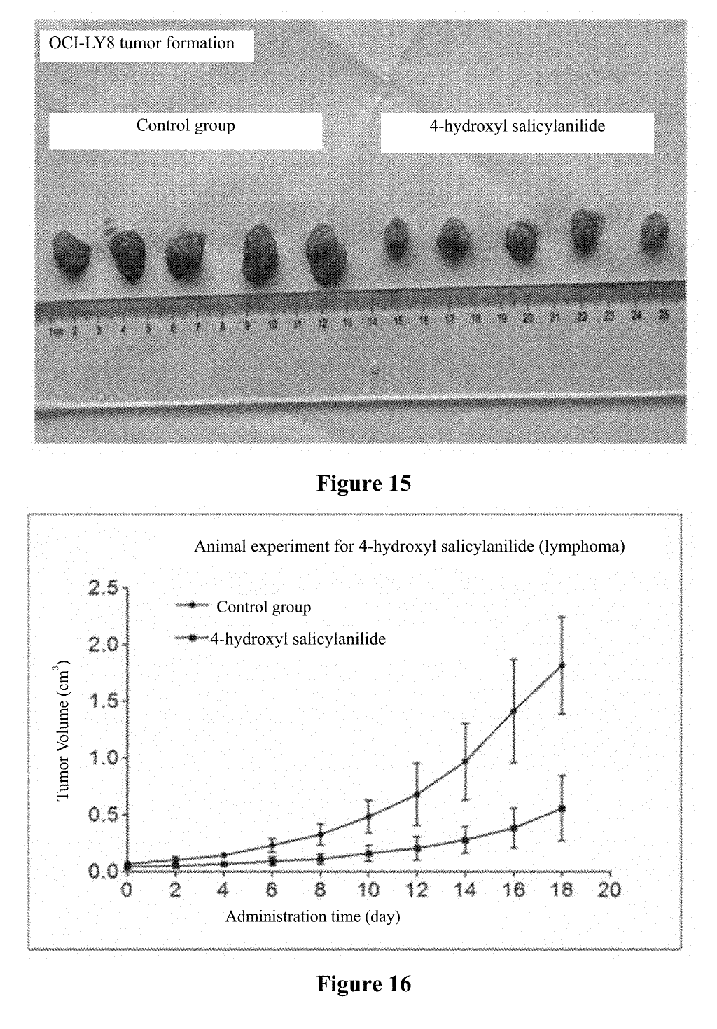

The experiment results are shown in FIG. 15 and FIG. 16.

Conclusion: 4-hydroxy salicylanilide can effectively inhibit the growth of lymphoma in nude mice.

The above description is only a preferred embodiment of the present invention, and it should be understood that those skilled in the art can make several improvements and supplements without departing from the method of the present invention. These improvements and supplements should also be considered to fall into the scope of the present invention.

1-7. (canceled)

8. A method for preventing or treating a tumor in mammal, which comprises a step of administrating 4-hydroxy salicylanilide or a pharmaceutically acceptable salt thereof to a subject in need, wherein the tumor is selected from the group consisting of lymphoma, and multiple myeloma.

9. The method of claim 8, wherein the subject is a human.

10. The method of claim 8, wherein the lymphoma is a non-Hodgkin's lymphoma.

11. The method of claim 10, wherein the non-Hodgkin's lymphoma is a B cell lymphoma.

12. The method of claim 11, wherein the B cell lymphoma is a diffuse large B cell lymphoma.

13. The method of claim 11, wherein 4-hydroxy salicylanilide or a pharmaceutically acceptable salt thereof is formulated into a pharmaceutical composition.

14. The method of claim 13 wherein the pharmaceutical composition comprises 4-hydroxy salicylanilide or a pharmaceutically acceptable salt thereof and a pharmaceutically acceptable carrier.

15. The method of claim 13, wherein the pharmaceutical composition is prepared into a tablet, granule, injection or capsule.

16. A method for preventing or treating a tumor in a subject, comprising a step of administrating 4-hydroxy salicylanilide or a pharmaceutically acceptable salt thereof to the subject, wherein the tumor is a solid tumor.

17. The method of claim 16, wherein the subject is a human.
